(19) United States
(12) Patent Application Publication
(10) Pub. No.: US 2017/0226588 A1
(43) Pub. Date: Aug. 10, 2017

(54) SYSTEMS AND METHODS FOR DNA AMPLIFICATION WITH POST-SEQUENCING DATA FILTERING AND CELL ISOLATION

(71) Applicant: CYNVENIO BIOSYSTEMS INC., Westlake Village, CA (US)

(72) Inventors: William M. STRAUSS, WESTLAKE VILLAGE, CA (US); Nathan GOODMAN, SEATTLE, WA (US)

(21) Appl. No.: 15/016,174

(22) Filed: Feb. 4, 2016

(51) Int. Cl. C12Q 1/68; G06F 19/22

(52) U.S. Cl. CPC C12Q 1/6886; G06F 19/22; C12Q 1/6806; C12Q 2600/156

(57) ABSTRACT

A heuristic filtering system and method are described for variant DNA within a heterogeneous cell sample. After ion semiconductor sequencing, the amplicons are processed through a series of filters designed to eliminate noise in the variants to provide a clearer set of variant results. Reports are generated, showing both the filtered results and the effects the filters had on the original data.

### BACKGROUND

It is important to accurately determine the presence of disease causing mutations in a patient, given the severity not only of the disease, but also of the treatment for such diseases (e.g., chemotherapy or radiation treatment). A method for determining the presence of such mutations can be performed by taking a tissue or fluid sample from the patient, then sequencing the sample looking for variants (mutations) in the DNA. However, there are factors in both sample procurement and sequencing that can lead to an abundance of false positive results that reduce the confidence level of the test results.

### SUMMARY

In a first embodiment, a method for detection of variant DNA in a heterogenous cell sample is described, the method comprising: sequencing the heterogenous cell sample from a subject, producing an input sequence; and applying a heuristic filter pipeline to the input sequence, producing an output report.

In a second embodiment, a method is described as the method of the first embodiment further comprising: sequencing a control cell sample from the subject, producing a control sequence.

In a third embodiment, a method is described as the method of the second embodiment wherein the heuristic filter pipeline further comprises at least one of: determining amplicons to be excluded; determining read positions to be excluded; and determining variants to be excluded.

These embodiments are exemplary and other embodiments are understood from the disclosure. One skilled in the art could conceive of further embodiments from the teachings herein.

### BRIEF DESCRIPTION OF DRAWINGS

The accompanying drawings, which are incorporated into and constitute a part of this specification, illustrate one or more embodiments of the present disclosure and, together with the description of example embodiments, serve to explain the principles and implementations of the disclosure.

FIG. 1 illustrates an exemplary Cluster filter application.

FIG. 2 illustrates an exemplary Global filter application.

FIGS. 3A and 3B illustrate an exemplary graph showing a reduction of noise due to the heuristic filtering system.

FIG. 4 illustrates an exemplary filtering engine.

FIG. 5 illustrates an example method of reporting DNA variants in a patient cell sample.

FIGS. 6A and 6B illustrate an example heuristic filter pipeline flowchart.

FIG. 7 illustrates an example of computer hardware for the heuristic filter pipeline.

### DETAILED DESCRIPTION

Genome sequencing is useful for detection and identification of disease mutations in cells, such as with cancer. Difficulties can arise in computer-aided sequencing when the biological sample, for example taken from a blood sample from a patient, contains a heterogeneous mixture of cell deoxyribonucleic acid (DNA).

Nucleic acid sequencing is a method for determining the exact order of nucleotides present in a given DNA or RNA molecule. Next-generation sequencing (NGS), also known as high-throughput sequencing, is a term used to describe a number of different modern nucleic acid sequencing technologies including Illumia™ sequencing, Roche 454™ sequencing, Ion torrent: Protein/PGM™ sequencing and SOLiD™ sequencing. These sequencing technologies allow one to sequence DNA and RNA quickly and cheaply compared to the previously used Sanger sequencing.

The term “nucleic acids” “polynucleotides” as used herein refer to biological molecules comprising a plurality of nucleotides. Exemplary nucleic acids include deoxyribonucleic acids (DNA) and ribonucleic acids (RNA), each synthesized from four different types of nucleotides, also called “bases”. The nucleotides for DNA include deoxy-adenosine (“A”), deoxy-thymidine (“T”), deoxy-cytosine (“C”), and deoxy-guanosine (“G”). The nucleotides for RNA include adenosine (“A”), uracil (“U”), cytosine (“C”) and guanosine (“G”). The nucleotides of a DNA or RNA are arranged in a particular order, referred to as the sequence of the DNA or RNA. The precise order of nucleotides, i.e. the four bases, within a DNA or RNA molecule is determined using nucleic acid sequencing methods.

In cases where a suspected disease or condition is concerned, targeted sequencing of specific genes or genomic regions is preferred. Compared to whole genome sequencing, which sequences an entire genome, targeted sequencing targets on a sequence segment of interest comprising one or more specific genes or genomic regions. Targeted sequencing generally yields higher coverage of genomic regions of interest and reduces sequencing cost and time.

“Amplicon sequencing” as used herein refers to a targeted sequencing method in which a discrete region of a genome is first amplified from the entire genome using PCR and the generated amplicons are used as templates for subsequent sequencing. Amplicon sequencing is typically used to investigate genetic variants in complex and heterogeneous samples. Sequencing can be carried out in a sample containing amplification products of a single amplicon. Alternatively, the sample can contain mixtures of multiple amplicons pooled together, as will be understood by a skilled person. Amplicon Sequencing is a method where multiple amplicons are pooled together and co-sequenced.

“Amplicons” as used herein are defined as replicated DNA (or ribonucleic acid—RNA) strands that are formed by polymerase chain reaction (PCR), ligase chain reactions (LCR), or other DNA duplication methods, where the strands are copies of a target region of a genome. In order to multiplex PCR amplification, each amplicon has to be unique and independent (no overlapping amplicons), which requires careful selection of the primers used to tag the regions to be amplified. Amplicons for sequencing have a length typically in the range between 100 bp and 500 bp.

The processing and sequencing of amplicons with different sequencing platforms can be flexible and allows for a range of experimental designs. A variety of options regarding design parameters can be selected, such as the length of amplicons, the number of amplicons pooled together, the number of reads desired for a given amplicon or a pool of amplicons, whether to read from one end (unidirectional sequencing) or both ends (bi-directional sequencing) of the amplicon and other factors identifiable to a skilled person in the art.

“Read” or “reads” used herein are defined as a sequenced range of DNA or RNA. A read can be a sequence that is output by a sequencing instrument, where the read attempts to match a range of DNA that was input to the instrument. Each set of reads maps to a particular amplicon, with a read being a sequence for the complete amplicon or, typically, a range of bases comprising a subset of the amplicon. The total set of reads in the input data for the filter pipeline can include multiple amplicons, each having multiple reads mapped to them. The range of the read lengths depends upon the primers chosen for a given library. The mapping of reads to an amplicon can be determined during alignment/assembly using a sequencing alignment tool, for example the Bowtie™ 2 read alignment tool from Johns Hopkins University (see “Fast gapped-read alignment with Bowtie 2” by Ben Langmead and Steven L. Saizberg, Nat Methods, Author manuscript; PMC Apr. 1, 2013).

In order to analyze libraries formed from heterogeneous mixtures of DNA (i.e., a mixture of different cells), rare sequencing events that contain a disease mutation, called herein the “signal”, must be differentiated or filtered from extraneous sequencing information, called herein the “noise”. A signal that is of the same order of magnitude as noise (e.g., a high frequency of DNA in the sample that is not being targeted for analysis) is difficult to interpret unless a specific filtering method is used to remove at least some of the noise.

There are at least two sources of noise in the sequencing pipeline. First, the DNA mixtures that are produced from input pellets (DNA or cell pellets) are complicated mixtures of cells and therefore any useful signal is diluted by DNA that has no informational content. A second source of noise is due to the specific sequencing technology employed. For example, sequencing noise or “machine” noise can be derived from an ion-to-bases sequencing process, for example with the Ion Torrent™ Personal Genome Machine (PGM™) platform. For example, ion detection sequencing that reads bases on pH detection is sensitive to homopolymers and will sometimes read a homopolymer chain as being one base too long or too short, particularly if the chain is long.

As used herein, “ion-to-bases” refers to ion semiconductor sequencing or ion detection sequencing, a method of sequencing DNA based on the detection of hydrogen ions that are released during the polymerization of DNA. This is a method of sequencing by synthesis, such that a complementary strand is built based on the sequence of the target strand.

Based upon empirical evidence, the machine noise contribution can be 5% to 10% or higher. Based upon the nature of the rare cell pellets recovered from a cell isolation platform, the required theoretical sensitivity needs to be on the order of about 1% to enable useful patient information to be reproducibly recovered from samples. Given that this sensitivity is not compatible with the noise characteristics of the sequencing platform, an informatics based sequence filtering strategy is required to reduce the noise below the required sensitivity (for example, 1%, or one cell in one hundred being a target cell). The noise in a sequencing pipeline can be reduced significantly by a heuristic filtration method.

The ability to distinguish a sequence variant (SNV) from a non-variant/reference genome requires sufficient sampling of the test sample to ensure a statistically valid result (i.e., a satisfactory degree of confidence in the results). For example, at the 1.0% threshold this translates to 20 informative (mutation bearing) reads per 2000 total reads. Cell-free DNA, however, may not have enough integrity to allow that many reads, so a lower threshold might be required, which in turn results in a lower level of confidence in the results. In addition to collecting a sufficient number of total reads, there are other considerations that affect the ability to call SNV's from sequencing tests. In order to call a sequence variant as a true mutation, confounding artifacts of the sequencing process must be excluded.

A sequence variant also known as mutations include deletions, insertions, substitutions and duplications of a single or multiple nucleotides and chromosome rearrangements such as translocation and inversion. A particular type of sequence variant indicates a genetic variation formed by single base pair substitution, called a point mutation.

Once the FASTQ files (i.e., text-based files containing sequences of reads produced from a genome sequencing procedure) are exported from the ion-to-bases conversion server they must be analyzed for sequence variants (SNVs). In order for this to be accomplished, a sequence alignment of the experimental files to the reference sequence must be accomplished. In order to perform an alignment of the FASTQ sequences to a human reference assembly, a sequence alignment software device is required. This alignment output is in a BAM format. The BAM format is a binary version of the SAM (Sequence Alignment/Map) tab delimited file alignment. Once an indexed BAM file has been produced and gapped, the actual alignment can be visualized if needed.

Despite the alignment of each FASTQ read to the reference sequence (an amplicon), there is still a chance that a given base will be in error due to the base calling or due to biological or machine noise. Thus a post-alignment software program for sequence analysis has been developed. This program is called the “heuristic filter pipeline”, a series of filtering steps that generates an SNV report from the FASTQ data. This SNV report can then be exported into the LIMS (Laboratory Information Management System) for patient reporting. An example heuristic filter algorithm is as below:
1) Review each amplicon for reads mapping to that amplicon. Exclude the entire amplicon (i.e. all of the reads mapped to that amplicon; as determined, for example, from an alignment/assembly process) from the results if the number of mapped reads is below a threshold value. A threshold of 2000 is typical, but lower thresholds, such as 500, can be set if the threshold excludes too many amplicons. (Amplicon Coverage filter).
2) Count the total variant base calls across all the reads for each position. If the number of variant base calls is below a threshold, exclude all SNV at that the position from the results. The threshold can be a percentage of variants for the reads (e.g., if less than 1% of the reads has a variant at that position, exclude the position from the results). (Variant Count filter).
3) Exclude any positions that have been marked in the database as having known problems (for example, as known from previous runs, or from external knowledge and added to the database by a user). (Exclusion filter).
4) Exclude any positions that have a number of reads below a threshold value (e.g., if a position is only found in under 2000 reads, exclude all SNVs at that position from the results). As with the Amplicon Coverage filter above, the threshold can be lowered if the higher value excludes too many positions. (Base Coverage filter).
5) Using a “case/control” model, compare the experimental sample DNA to a negative control DNA for each SNV. Any candidate SNV of the experimental sample must not be present in the negative control. (Case/Control filter).
6) Determine the position of the SNV relative to each end of the read. Any candidate SNV must be greater than a set value (for example, 11) nucleotides from either end of a trimmed read. This is based on idea that hits near the ends of each sequence are unreliable. (End-of-Read filter).
7) Evaluate the position nᵢ in the amplicon for homopolymers. Any candidate SNV shall not be found within a preexisting homopolymer track greater or equal to a set value (for example, 4) nucleotides relative to the reference. This is because ion-to-bases resequencing has difficulty reading strings of homopolymers, especially long ones. (Homopolymer filter)
8) Evaluate the region surrounding SNV (i.e., at position nᵢ±δ_{c}) on each read containing a variant for adjacent or clustered variants. Within a particular read there cannot be additional substitutions, regardless of base type, in the delimited region (for example, within 100 bases/positions; or as another example, within the entire amplicon length). Optionally, this step could also be combined with the Variant Count filter, wherein the Variant Count filter can be run (or re-run) with the set of reads remaining after reads are removed with the Cluster filter. For example, suppose the variant cutoff is 1%, and there is an initial count of 4000 reads of which 41 had a variant at position 100. Now suppose the Cluster filter step removes 1000 reads, leaving 3000 remaining reads. If 30 or more of the remaining 3000 reads still have the variant at position 100, the variant passes the step and is retained. If, however, fewer than 30 reads have the variant, the variant fails the step and is removed from further consideration in the pipeline. This could result in some variants that were originally removed by the Variant Count filter to now pass the Variant Count filter. This can be addressed one of two ways: the variants can be re-introduced into the results, optionally with them being re-run through the pipeline to be checked against any filters they would have missed in the previous run; or the pipeline can be run as exclude-only, so that the re-run Variant Count filter does not re-introduce previously failing variants, but only excludes previously passing variants. (Cluster filter)
9) Evaluate the region surrounding SNV (i.e., at position nᵢ±δ_{g}) for all reads of an amplicon (or, alternatively, for a subset of reads) for additional variants and exclude the SNV if too many additional variants not already excluded by the Amplicon Coverage filter and with the same non-reference base are found (i.e. beyond a threshold value, even a threshold of 0 where just one additional variant of that same base would be considered too many). An example value for δ_{g} is 5. (Global filter)
10) Determine which variants are reportable based on knowledge of clinical ramifications. (Report filter).
11) Post the heuristic filter pipeline hit list analysis.

The filters can be applied in any order, and in any combination (i.e., not all filters need to be used). The inclusion of and thresholds used by the various filters can depend upon the nature of the input data and the sources of noise present in the DNA acquisition and sequencing process. Each filter step can also record a percentage of pass and/or fail rate for that filter as a threshold to determine if the filter should be applied to the results (for example, if the number of amplicons failing the Amplicon Coverage filter is too high—or equivalently if the number of amplicons passing the Amplicon Coverage filter is too low—then the amplicons that would be excluded from the Amplicon Coverage filter are not excluded). This would create a controllable tolerance level for the filter in question, allowing a filter be more permissive for batches that would otherwise have too few remaining SNVs after filtering.

“Noise”, as used herein, includes false positive and unreliable results from any source, internal or external to the system, or data that is not clinically significant. “Signal”, as used herein, includes highly reliable results that a user is trying to analyze.

Case/control can include comparing sequences from the patient's sample (e.g., blood to be analyzed) and a germatic control sample (e.g., patient's normal/unmutated tissue).

In an example library, for a three minute assembly the post-assembly process adds about one and a half minutes to the process.

In addition to the hit/miss statistics, the hit list analysis can include details of why each removed hit was filtered out. The specific filter (Case/control, End-of-read, Cluster, etc.) that removed the hit can be listed next to the hit for analysis of the noise of the system.

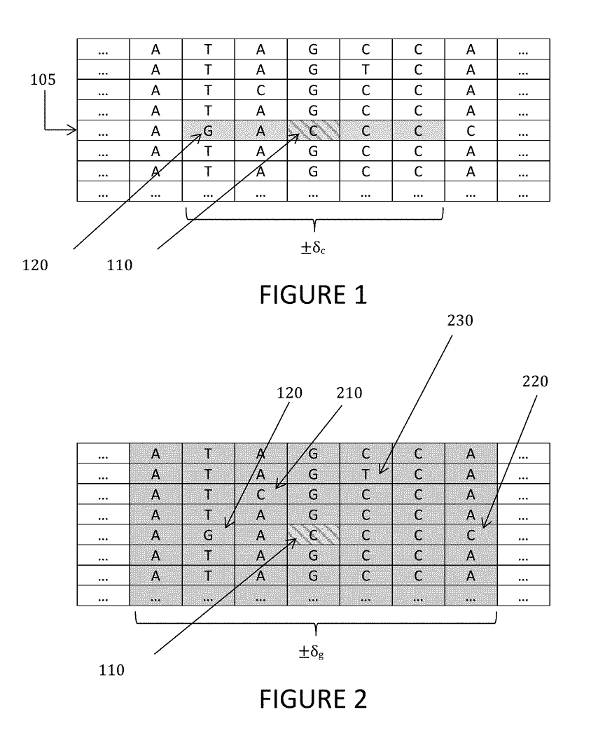

FIG. 1 illustrates an exemplary Cluster filter application. For a genomic sequence stack, with rows of reads stacked so that each column being a particular base location (position) in genome, a particular SNV (110) is analyzed. A region is defined ±δ_{c} bases to the left and right of the SNV (110) at the read containing the SNV (105). If there are any other variants not already filtered out with the Amplicon Coverage filter found in this region, the SNV (110) is filtered out of the results. As shown in the example, there is an additional variant (120) that would cause the Cluster filter to filter out the SNV (110). Alternatively, the entire read (a row in FIG. 1) that the SNV (110) is located in could be removed from consideration from a subsequent Variant Count filtering step, and the SNV (110) would be removed from the final results if it fails the Variant Count filtering with the reads removed due to Cluster filter failures removed from consideration.

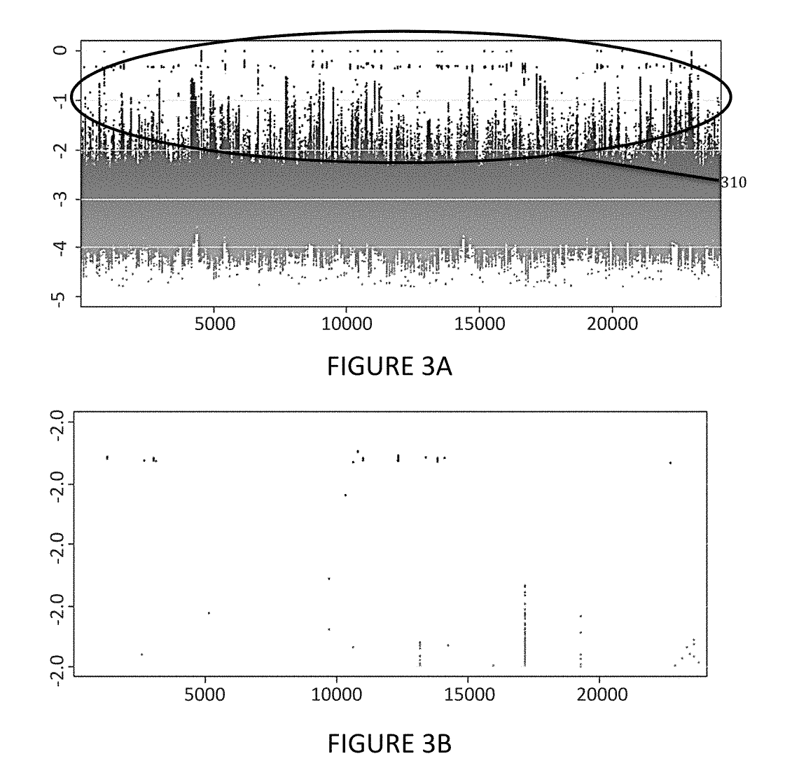

FIG. 2 illustrates an exemplary Global filter application. For a genomic sequence stack, with rows of reads stacked so that each column being a particular base location (position) in genome, a particular SNV (110) is analyzed. A region is defined as ±δ_{b} bases to the left and right of the SNV (110) for all (or a subset of all) reads. If there are any other variants not already filtered out with the Amplicon Coverage filter and with the same non-reference base as the SNV found in this region, the SNV is filtered out of the results. As shown in the example, there are there additional variants (210 and 220) that would cause the Global filter to filter out the SNV (110). Note that variants (120 and 230) not matching the SNV (110) base type are not considered to be “additional variants” for the Global filter—only matching bases are considered. In an alternative embodiment, the Global filter can consider all variants (120, 210, 220, and 230) when determining if there are additional variants in the region. If the Cluster filter, as shown in FIG. 1, is applied before the Global filter, the SNV (110) could be filtered out by the Cluster filter before the application of the Global filter due to a variant (120) also being in the Cluster filter range (±δ_{c}). While this Global filter shows the entire list of reads, it could also be performed for a subset of the reads.

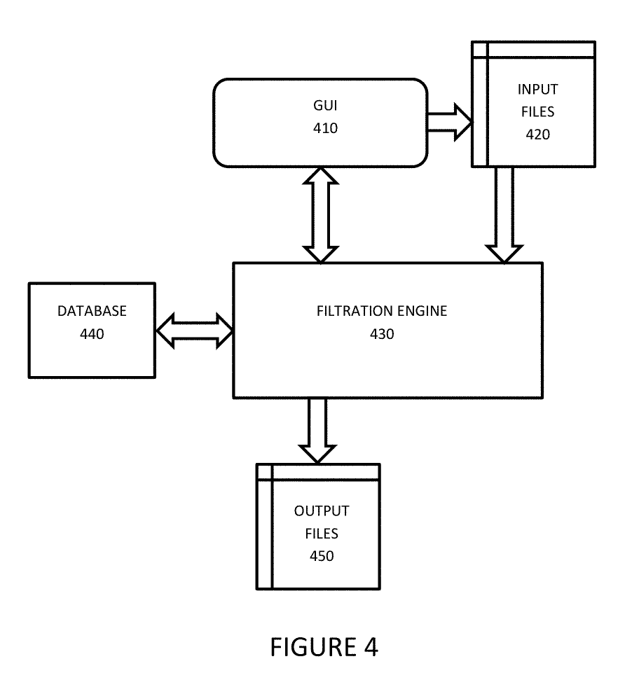

FIGS. 3A and 3B illustrate an exemplary graph showing a reduction of noise due to heuristic filtering. FIG. 3A illustrates variant rate (vertical axis—logarithmic scale) for each genome position (horizontal axis) found in an ion-to-bases process (pre-filter-pipeline). The y-axis shows the variant rate, i.e. the fraction of reads that have a non-reference base at a given position. The rate for this graph is expressed in log base 10 units. The maximum value of 0 is equivalent to a rate of 1.00—i.e. 100% of reads have non-reference base at the position; a value of −1 means 10% reads are non-reference, a value of −2 means 1% are non-reference; and so on. The 0 to −2 range (310) corresponds to positions at which more than 1% of reads are non-reference, and it is within this range that positions are found that can be used for calling variants if given a 1% tolerance level.

As it can be shown by FIG. 3A, there are many variant hits, even in the region above the −2 mark (310) which represents a significant amount of noise interfering with the signal data. FIG. 3B illustrates the same data in the 0 to −2 range (310) after going through heuristic filtering. With the removal of noise, significant variants are more clearly identified.

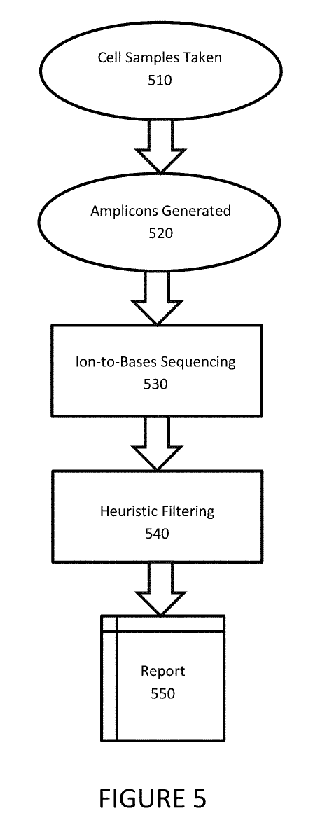

FIG. 4 illustrates an exemplary SNV filtering engine. A user can control the filtering engine (430) through a user interface (410), for example a graphical user interface, which allows in input of files (420) to be processed by the engine (430). The filtering engine (430) takes the input files (420), for example an ion-to-bases sequencing FASTQ results file, and applies heuristic filtering on the files (420) to produce output files (450) which can include post-filtered variant identification and data related to the filtering process, such as identifying variants identified in the input files (420) that were filtered out by the filtering engine (430). A database (440) can be connected to the filtering engine (430) for storage of data for the output files (450). Control variables for the filtering engine (430) can be input at the user interface (410) or be included in parameter files included in the input files (420).

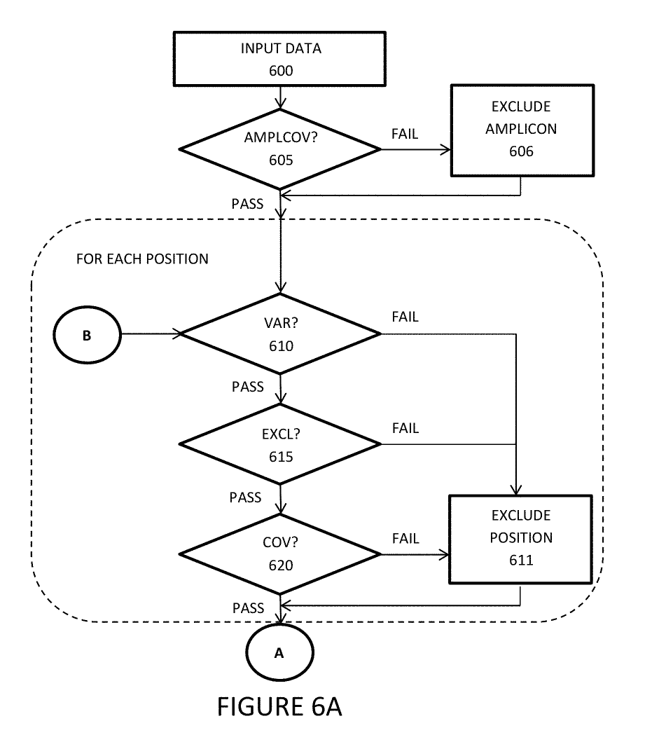

FIG. 5 illustrates a method of reporting DNA variants in a patient cell sample. A sample of cells are taken (510) from a subject. For example, a blood or biopsy sample can be taken from a cancer patient in order to detect disease variants in the patient's DNA. Amplicons are generated (520) from the sample, for example using a polymerase chain reaction (PCR) process. A negative control sample, such as a baseline healthy (unmutated) sample from the subject, can also be amplified to aid the filtering (540) process. These amplicons can then be sequenced (530) by an ion-to-bases sequencing process. The results of the sequencing can then be filtered (540) by the heuristic filtering process described herein. The filtering (540) can then produce a report (550) that identifies highly likely locations of variants (mutations) within the genomic sample. The report (550) can also include information regarding which results were removed during filtration (540) and which type of filter was used to remove the result.

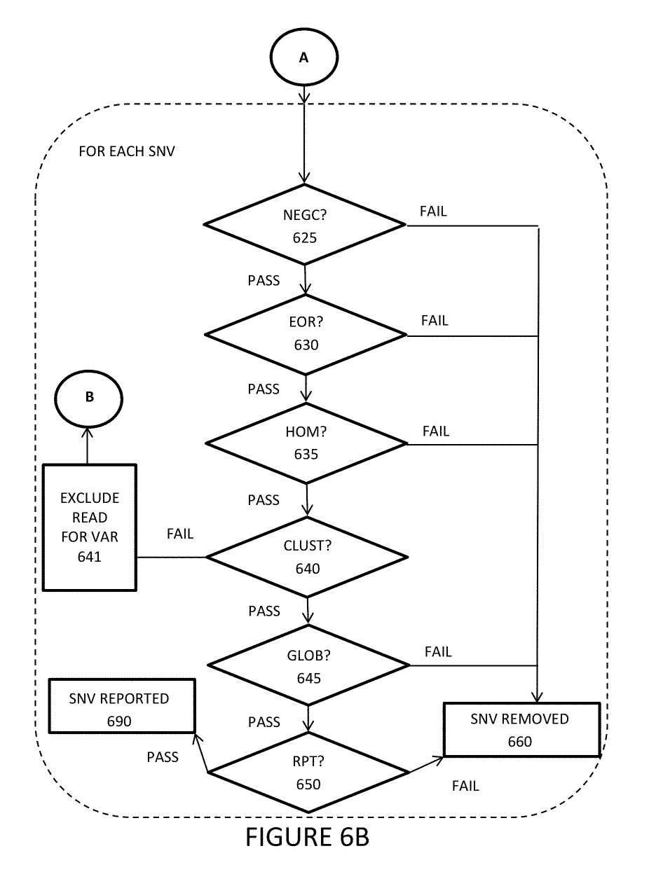

FIGS. 6A and 6B illustrate an example heuristic filter flowchart for filtering non-synonymous SNV candidate data (600). The filter steps can each remove SNVs individually (660), or remove entire positions (611) or amplicons (606), for the output report. The removals themselves can be recorded, however, for filtering analysis. See Table 1 for an example filtering analysis report.

FIG. 6A shows example filter steps that exclude amplicons and positions from the output report. The data is entered (600) to the pipeline, and can be filtered to determine amplicon coverage (605). If the number of reads for a given amplicon falls below a threshold value, then the reads for the entire amplicon are excluded (606). The positions can then be considered. The variant base call count (610) for a given position can be considered and, if the number (or ratio) of variants found at that position falls below a threshold value (for example, 1% of all reads), then that position is excluded (611). The pipeline can also filter out positions that are known to give unreliable variant counts (615). Also, positions that have an insufficient number of reads (620) can be excluded as unreliable data (611). The positions can be reviewed incrementally, with each position being run through the filters on a position-by-position basis until it is excluded or passes all filters, or each filter can in turn consider all of the positions that were not excluded by previous filters.

FIG. 6B shows a continuation of the filter pipeline from FIG. 6A, where individual SNVs are filtered (660) from the final variant report. If a negative control sequence is available, the SNV can be compared to the negative control (625). If the SNV is also found in the negative control, then that SNV can be excluded (660). The SNVs that appear too close to either end of a read (630) can also be excluded (660). SNVs that appear in a preexisting homopolymer track at or above a certain length (635) can also be excluded (660) as being unreliable data. If there are other variants too close (i.e., within a range, such as) to the SNV on that read, then that read is excluded (641) from the variant rate calculation (610), which could result in the SNV being excluded by an exclusion of that position (611). Also, if there are too many (which could mean “any”) variants in any read (or a subset of reads) that is too close to the position (for example, within 5 positions) of the SNV (645), then that SNV can be excluded (660) as unreliable. Additionally, any SNVs that are considered not reportable due to knowledge of clinical ramifications (650) (e.g., variants that are not considered relevant to the particular disease being screened for) can be excluded (660) as irrelevant. Any SNV that remain after the application of the filters can then be used (690) to form an analytical report. As with the filters based on position, the filters can either iteratively consider each SNV until that SNV is excluded, or each filter can process the total SNVs that have not been excluded by previously applied filters.

TABLE 1
Example Filter Report

| run | pat_id | filter | chr | coordinate | aref | avar | coverage | var_count | effect |
|---|---|---|---|---|---|---|---|---|---|
| 302 | LB517 | NON | 9 | 133747505 | T | C | 10326 | 475 | — |
| 302 | LB517 | NON | 9 | 133747506 | C | T | 10317 | 274 | — |
| 302 | LB517 | NON | 9 | 133747507 | C | T | 10314 | 482 | — |
| 302 | LB517 | EOR | 14 | 105241519 | T | C | 17603 | 1134 | NS |
| 302 | LB5017 | NON | 2 | 29432625 | C | A | 5744 | 786 | — |
| 302 | LB5017 | GLOB | 5 | 112175211 | T | A | 2503 | 32 | NS |
| 302 | LB5017 | GLOB | 5 | 112175216 | G | A | 2506 | 71 | NS |

Table 1 shows a portion of an example filter report. For a given sequencing run (run) for a given patient (pat_id), variants (avar) are shown relative to the reference base (aref) they substitute with the variant location identified by chromosome number (chr) and gene coordinate (coordinate). The total base coverage (coverage) and variant count (var_count) for the variant is given. A filter report field (filter) reports whether the variant was not filtered by the heuristic filter (value of NON) or, if it was filtered, which filter removed the variant from the final results (e.g., EOR for end-of-read filter, GLOB for global filter, etc.). Another field (effect) reports other effects that can determine scoring of the variant, such as being non-synonymous (value NS). The report can include further information, such as the type of run (e.g., germ line run), base counts at that position, percent variation, deletion counts, gene identification, transcript identification, protein change, complimentary DNA (cDNA) change, or Catalogue of Somatic Mutations in Cancer (COSMIC) identification.

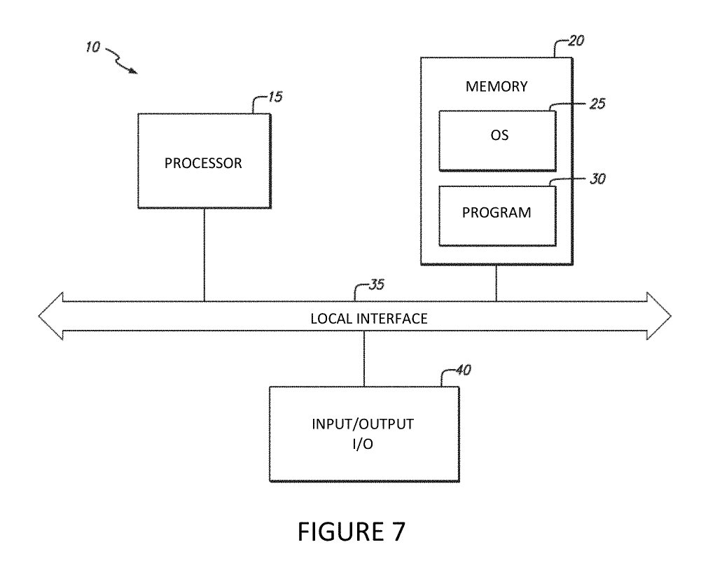

FIG. 7 is an exemplary embodiment of a target hardware (10) (e.g., a computer system) for implementing the embodiment of FIGS. 1 to 6B. This target hardware comprises a processor (15), a memory bank (20), a local interface bus (35) and one or more Input/Output devices (40). The processor may execute one or more instructions related to the implementation of FIGS. 1 to 6B and as provided by the Operating System (25) based on some executable program (30) stored in the memory (20). These instructions are carried to the processor (15) via the local interface (35) and as dictated by some data interface protocol specific to the local interface and the processor (15). It should be noted that the local interface (35) is a symbolic representation of several elements such as controllers, buffers (caches), drivers, repeaters and receivers that are generally directed at providing address, control, and/or data connections between multiple elements of a processor based system. In some embodiments the processor (15) may be fitted with some local memory (cache) where it can store some of the instructions to be performed for some added execution speed. Execution of the instructions by the processor may require usage of some input/output device (40), such as inputting data from a file stored on a hard disk, inputting commands from a keyboard, inputting data and/or commands from a touchscreen, outputting data to a display, or outputting data to a USB flash drive. In some embodiments, the operating system (25) facilitates these tasks by being the central element to gathering the various data and instructions required for the execution of the program and provide these to the microprocessor. In some embodiments the operating system may not exist, and all the tasks are under direct control of the processor (15), although the basic architecture of the target hardware device (10) will remain the same as depicted in FIG. 7. In some embodiments a plurality of processors may be used in a parallel configuration for added execution speed. In such a case, the executable program may be specifically tailored to a parallel execution. Also, in some embodiments the processor (15) may execute part of the implementation of FIGS. 1 to 6B and some other part may be implemented using dedicated hardware/firmware placed at an Input/Output location accessible by the target hardware (10) via local interface (35). The target hardware (10) may include a plurality of executable programs (30), wherein each may run independently or in combination with one another.

A number of embodiments of the disclosure have been described. Nevertheless, it will be understood that various modifications may be made without departing from the spirit and scope of the present disclosure. Accordingly, other embodiments are within the scope of the following claims.

The examples set forth above are provided to those of ordinary skill in the art as a complete disclosure and description of how to make and use the embodiments of the disclosure, and are not intended to limit the scope of what the inventor/inventors regard as their disclosure.

Modifications of the above-described modes for carrying out the methods and systems herein disclosed that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

It is to be understood that the disclosure is not limited to particular methods or systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

What is claimed is:

1. A method for detection of variant DNA in a heterogenous cell sample, the method comprising:
sequencing the heterogenous cell sample from a subject, producing an input sequence; and
applying a heuristic filter pipeline to the input sequence, producing an output report.

2. The method of claim 1, further comprising:
sequencing a control cell sample from the subject, producing a control sequence.

3. The method of claim 2, wherein the heuristic filter pipeline further comprises at least one of:
determining amplicons to be excluded;
determining read positions to be excluded; and
determining variants to be excluded.

4. The method of claim 3, wherein the heuristic filter at least comprises said determining the amplicons to be excluded, and said determining the amplicons to be excluded comprises counting the number of reads mapped to each amplicon and excluding each amplicon that has a number of mapped reads below a threshold value.

5. The method of claim 3, wherein the heuristic filter pipeline at least comprises said determining the read positions to be excluded, and said determining the read positions to be excluded comprises at least one of:
excluding each position that has a number or percentage of variant base calls below a variant count threshold;
excluding read each position that has been identified in a database to be excluded; and
excluding each position that is only present in a number of reads below a base coverage threshold.

6. The method of claim 3, wherein the heuristic filter pipeline at least comprises said determining the variants to be excluded, and said determining the variants to be excluded comprises at least one of:
excluding each variant that is found in a negative control sequence at that variant's position;
excluding each variant that is found within an end of read threshold range of an that variant's corresponding read;
excluding each variant that is within a homopolymer having a length equal to or greater than a homopolymer threshold;
excluding each read that contains any variant that has another variant within a cluster threshold range on that read;
excluding each variant, each of said each variant being at a corresponding variant position, that has over a variant threshold number of other variants within a global threshold range of the corresponding variant position on any read; and
excluding each variant that is determined to be excludable based on clinical ramifications.

7. The method of claim 4, wherein the threshold value is a value from 500 to 2000.

8. The method of claim 5, wherein the variant count threshold is 1% of the number of reads containing that position.

9. The method of claim 5, wherein the base coverage threshold is a value from 500 to 2000.

10. The method of claim 6, wherein the end of read threshold range is 11.

11. The method of claim 6, wherein the homopolymer threshold is 4.

12. The method of claim 6, wherein the cluster threshold range is 100.

13. The method of claim 6, wherein the variant threshold is 0 and the global threshold range is 5.

14. The method of claim 1, further comprising posting the output report.

15. The method of claim 1, wherein the output report includes a report of candidate variants that the heuristic filter removed from an output result of variants.

16. The method of claim 1, wherein the sequencing comprises ion-to-bases sequencing.

17. A computer system comprising:
at least one processor and memory configured to perform:
generation of a user interface;
file input;
the method of claim 1; and
file output.

18. The system of claim 17, further comprising a database.

19. The system of claim 18, wherein the database is a relational database.

20. The method of claim 1, further comprising procuring the heterogenous cell sample from the subject.

21. The method of claim 6, further comprising excluding each position that has a percentage of variant base calls below a variant count threshold for all reads not excluded by said excluding each read that contains any variant that has another variant within a cluster threshold range on that read
